(19) United States
(12) Patent Application Publication
(10) Pub. No.: US 2018/0206762 A1
(43) Pub. Date: Jul. 26, 2018

(54) SLEEP APNEA THERAPY ENHANCEMENT METHOD AND APPARATUS

(71) Applicant: Intel Corporation, Santa Clara, CA (US)

(72) Inventor: Jinshi Huang, Fremont, CA (US)

(21) Appl. No.: 15/415,740

(22) Filed: Jan. 25, 2017

(51) Int. Cl. A61B 5/08; A61B 5/024; A61B 5/00; A61B 5/087

(52) U.S. Cl. CPC A61B 5/08; A61B 5/0873; A61B 5/6824; A61B 5/02405

(57) ABSTRACT

Apparatuses, methods and storage medium associated with enhancing sleep apnea therapy are disclosed herein. In embodiments, an apparatus may comprise one or more light sources to project light onto a user; one or more optical sensors to sense light reflected off the user; and an analyzer coupled with the one or more optical sensors to receive, process and analyze the sensed light data for at least features of heart rate variability signals to determine an apnea condition of the user; wherein the determined apnea condition of the user is used to control an APAP device for the user. Other embodiments may be described and/or claimed.

### TECHNICAL FIELD

The present disclosure relates to the field of sleep apnea and wearable technologies. More particularly, embodiments of the present disclosure relate to sleep apnea therapy enhancement method and apparatus that includes usage of wearable devices.

### BACKGROUND

The background description provided herein is for the purpose of generally presenting the context of the disclosure. Unless otherwise indicated herein, the materials described in this section are not prior art to the claims in this application and are not admitted to be prior art by inclusion in this section.

Sleep apnea is a sleep disorder characterized by pauses in breathing or instances of shallow breathing during sleep. The lack of oxygen in the body due to sleep apnea can cause long-term health problem including high blood pressure, heart disease, stroke, diabetes and depression. This problem has affected the lives of millions of people around the world. One of the most common therapies to treat the sleep apnea is by using a continuous positive airway pressure (CPAP) device, which splints the patient's airway open during sleep by means of continuous, pressurized air.

An improved version of CPAP is the automatic positive airway pressure (APAP) device, where algorithms are used to sense the subtle changes in the patient's breathing and accordingly adjust the air pressure to the patient during sleep. However, until recently, the APAP machine only senses the conditions in the air flow, such as pressure, temperature and humidity in the tubing. This scheme has limitations as it has no information about the direct effects on the patient.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example, and not by way of limitation, in the figures of the accompanying drawings.

FIG. 1 illustrates an overview of the sleep apnea therapy enhancement technology of the present disclosure, according to various embodiments.

FIG. 2 illustrates an example wrist worn device incorporated with the sleep apnea therapy enhancement technology of the present disclosure, according to various other embodiments.

FIG. 3 illustrates an example of the recorded Electrocardiography (ECG), chest respiratory effort signals (Resp C) and, abdominal respiratory effort signals (Resp A), oronasal airflow (Resp N) and oxygen saturation (SpO2), and corresponding heart rate variability as measured by the R-R intervals (T Penzel, G B Moody, R G Mark, A L Goldberger, J H Peter. The Apnea-ECG Database. Computers in Cardiology 2000; 27:255-258).

FIG. 4 illustrates another example of the recorded Electrocardiography (ECG), chest respiratory effort signals (Resp C) and, abdominal respiratory effort signals (Resp A), oronasal airflow (Resp N) and oxygen saturation (SpO2), and the corresponding heart rate variability as measured by the R-R interval (T Penzel, G B Moody, R G Mark, AL Goldberger, J H Peter. The Apnea-ECG Database. Computers in Cardiology 2000; 27:255-258).

FIG. 5 illustrates an example process for processing optical sensor data, according to various embodiments.

FIG. 6 illustrates a component view of an example wearable device suitable for practicing the present disclosure, according to various embodiments.

FIG. 7 illustrates a component view of an example APAP device for practicing the present disclosure, according to various embodiments.

FIG. 8 illustrates a storage medium having instructions for practicing methods described with references to FIGS. 1-7, according to various embodiments.

### DETAILED DESCRIPTION

Apparatus, method and storage medium associated with enhancing sleep apnea therapy are disclosed herein. In embodiments, a wearable device, such as a wrist-worn device, with heart rate monitoring and sleep tracking functions may be worn by a user, and used to enhance an APAP device, in its provision of sleep apnea therapy to the user. Heart rate variability (HRV) data, together with other sleep tracking data, may be collected, processed and/or analyzed by the wearable device to determine an apnea condition of a user. The determined apnea condition of the user (optionally including the heart rate variability and sleep pattern data) may be reported to the APAP device. In response, the APAP device may adjust the machine settings based at least in part on the determined sleep apnea condition (HRV and/or sleep patterns), in addition to other sensor data (such as air pressure, humidity and temperature, etc. of the air flow provided to the user/patient).

Medical research has shown that HRV which is defined as the variation in time interval between heartbeats, manifests very different patterns for persons with and without sleep apnea. By taking into account the direct effects on the user/patient, the APAP device may be enhanced with increase accuracy of the device setting, and in turn, improve the effectiveness of the sleep apnea therapy provided to the user/patient.

In the following detailed description, reference is made to the accompanying drawings which form a part hereof wherein like numerals designate like parts throughout, and in which is shown by way of illustration embodiments that may be practiced. It is to be understood that other embodiments may be utilized and structural or logical changes may be made without departing from the scope of the present disclosure. Therefore, the following detailed description is not to be taken in a limiting sense, and the scope of embodiments is defined by the appended claims and their equivalents.

Aspects of the disclosure are disclosed in the accompanying description. Alternate embodiments of the present disclosure and their equivalents may be devised without parting from the spirit or scope of the present disclosure. It should be noted that like elements disclosed below are indicated by like reference numbers in the drawings.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiment. Various additional operations may be performed and/or described operations may be omitted in additional embodiments.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B and C).

The description may use the phrases “in an embodiment,” or “in embodiments,” which may each refer to one or more of the same or different embodiments. Furthermore, the terms “comprising,” “including,” “having,” and the like, as used with respect to embodiments of the present disclosure, are synonymous.

As used herein, the term “module” may refer to, be part of, or include an Application Specific Integrated Circuit (ASIC), an electronic circuit, a processor (shared, dedicated, or group) and/or memory (shared, dedicated, or group) that execute one or more software or firmware programs having machine instructions (generated from assembler instructions or compiled from higher level language instructions), a combinational logic circuit, and/or other suitable components that provide the described functionality.

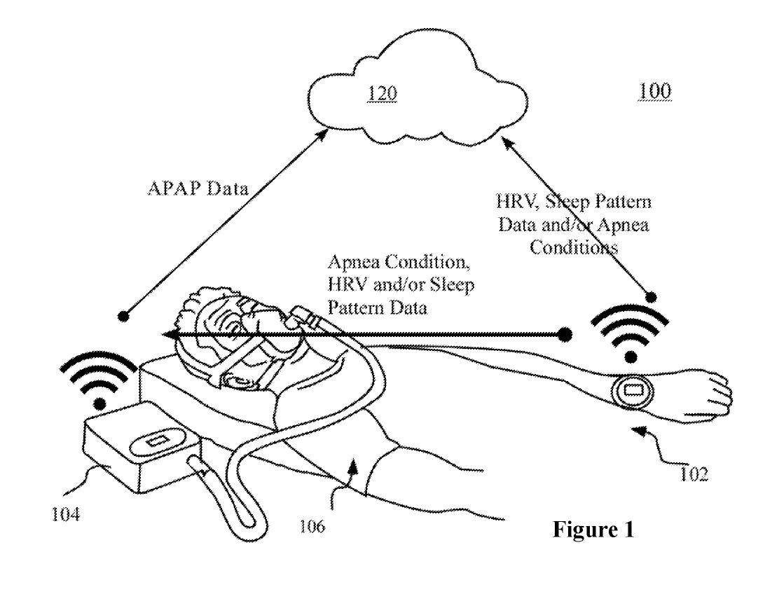

Referring now to FIG. 1, wherein a sleep apnea therapy system incorporated with the enhancement technology of the present disclosure, according to various embodiments, is illustrated. As shown, in various embodiments, sleep apnea system 100 may include wearable device 102 (which e.g., may be a wrist worn device) incorporated with the sleep apnea therapy technology of the present disclosure, to complement APAP device 104, to enhance the sleep apnea therapy provided by APA device 104 to user 106. User 106 may also be referred to as “patient,” or other like terms.

In embodiments, to facilitate enhancement of the sleep apnea therapy provided by APAP device 104, wearable/wrist worn device 102 may be configured to sense and derive heart rate variability (HRV) and other sleep related data of user/patient 106, as well as determine an apnea condition of the user/patient, based at least in part on the HRV and/or sleep pattern data, and provide the determined apnea condition (and optionally, the HRV and/or sleep pattern) to APAP device 104. APAP device 104 may be complementarily configured to receive the determined apnea condition of user/patient 106, and adapt its settings in provision of an airflow based on the received apnea condition of user/patient 106 to provide continuous positive airwave pressure to user/patient 106.

In embodiments, user/patient 106 may be determined to have a severe, moderate, mild apnea or no apnea condition. The detection of the sleep apnea and its severity can be performed by analyzing the HRV time series. Typical methods include calculating the power spectrum of the frames of the HRV time series, filtering the spectrum by different band-pass filters, and comparing the energy in the low frequency band (0.002 Hz to 0.5 Hz), the very low frequency (0.01 Hz to 0.05 Hz).

In embodiments, the HRV and/or the sleep pattern data may also be provided to the APAP device 104 to facilitate fine grain adaptation of its settings in provision of the airflow based on the HRV and/or the sleep pattern data, to provide continuous positive airwave pressure to user/patient 106.

Further, in embodiments, wearable/wrist worn device 102 and/or APAP device 104 may also be configured to provide the HRV and other sleep related data (as well as determined apnea conditions) and APAP data to one or more remote servers at a cloud computing service 120 for further analysis. The data provided by the wearable device may include the time series of the heart rate measurements and sleep tracking data (toss-and-turns, deep or light sleep, rapid eye movement, etc.). The data provided by the APAP machine can include sleep apnea statistics for hypopnea, obstructive apnea and clear airway apnea, as well as the machine setting such as pressure, humidity and temperature.

These and other aspects of the sleep apnea therapy enhancement technology of the present disclosure will be further described with references to the remaining Figures.

But before further describing the sleep apnea therapy enhancement technology of the present disclosure, it should be noted that while for ease of understanding, wearable device 102 is illustrated as a wrist worn device in FIG. 1, in alternate embodiments, as will be apparent from the description to follow, wearable device 102 may be other wearable devices, such as head worn devices, chest worn devices, arm worn devices, thigh worn device, or leg worn devices.

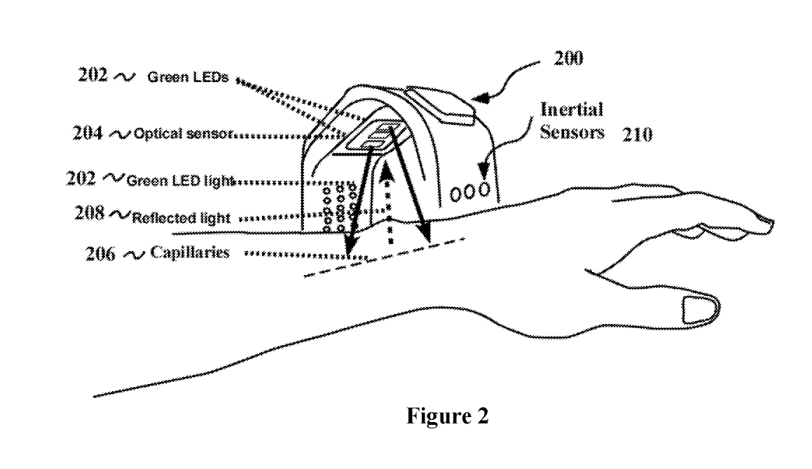

Referring now to FIG. 2, wherein an example wrist worn device incorporated with the sleep apnea therapy enhancement technology of the present disclosure, according to various other embodiments, is illustrated. As shown, in embodiments, wrist worn device 200 may include a plurality of light emitting diodes (LED) configured to emit light onto capillaries 206 of the user/patient, e.g. capillaries 206 at the wrist of the user/patient, in the case of wrist worn device 200. In embodiments, LEDs 202 may be green LEDs or other colors. Additionally, wrist worn device 200 may be configured with one or more optical sensors 204 to sense light 208 reflected off capillaries 206, and generate optical sensor data indicative of sensed light 208.

In embodiments, in addition to optical sensors 204, wrist worn device 200 may be configured with one or more inertial sensors 210 to collect various motion data that are indicative of the sleep pattern of the user/patient. Example of inertial sensors may include e.g., accelerometer and gyro sensors configured to sense motion of the user/patient to indicate the soundness or restlessness of the user/patient's sleep. It also senses the awakeness of the user/patient during the night.

Further, wrist worn device 200 may be configured to process and analyze optical and/or inertia data to determine an apnea condition of the user/patient. In embodiments, wrist worn device 200 may be configured to process the optical data to derive the heart rate variability of the user/patient, which substantially correlates with the nasal pressure experienced by the user/patient.

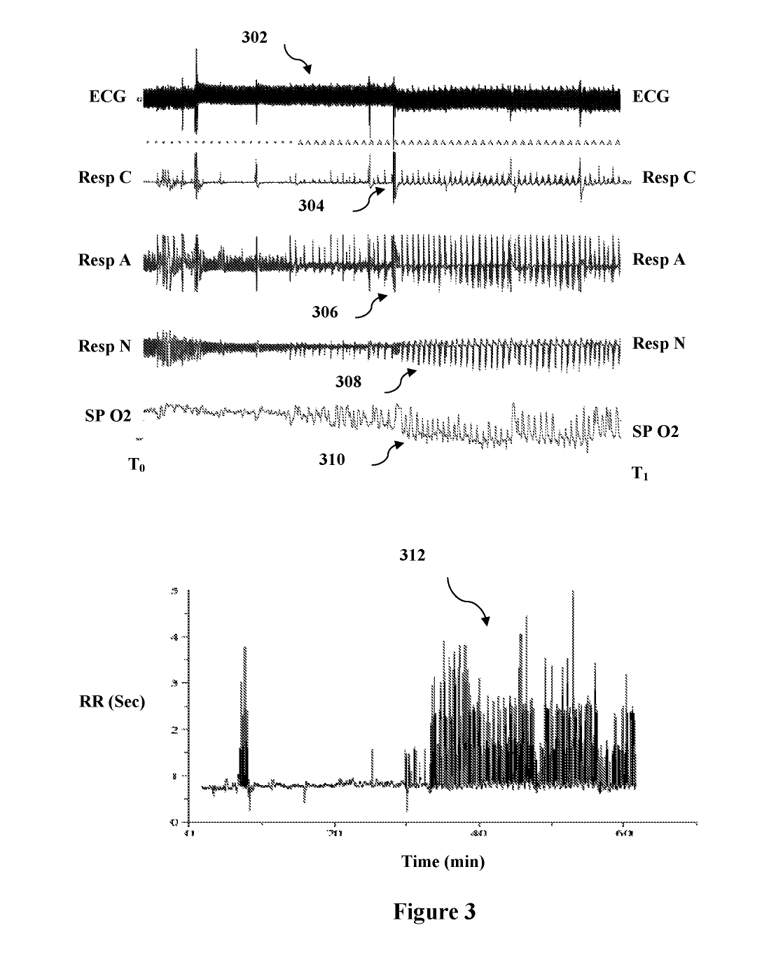

FIG. 3 illustrates an example of the recorded Electrocardiography (ECG), chest respiratory effort signals (Resp C) and, abdominal respiratory effort signals (Resp A), oronasal airflow (Resp N) and oxygen saturation (SpO2), and corresponding heart rate variability as measured by the R-R intervals (T Penzel, G B Moody, R G Mark, A L Goldberger, J H Peter. The Apnea-ECG Database. Computers in Cardiology 2000; 27:255-258).

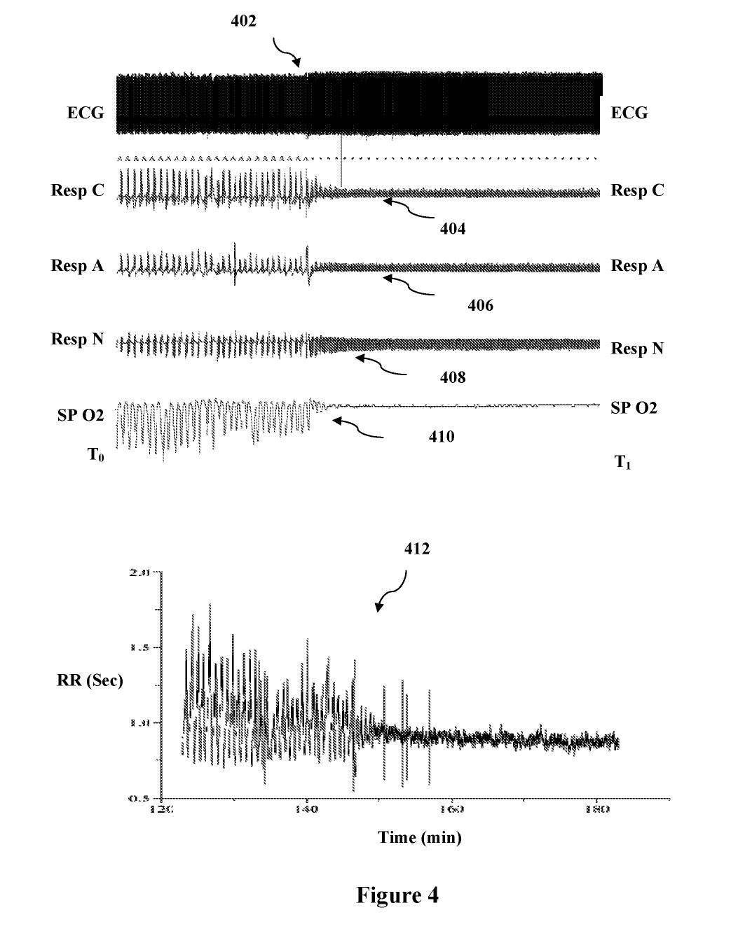

FIG. 4 illustrates another example of the recorded Electrocardiography (ECG), chest respiratory effort signals (Resp C) and, abdominal respiratory effort signals (Resp A), oronasal airflow (Resp N) and oxygen saturation (SpO2), and corresponding heart rate variability as measured by the R-R intervals (T Penzel, G B Moody, R G Mark, A L Goldberger, J H Peter. The Apnea-ECG Database. Computers in Cardiology 2000; 27:255-258.).

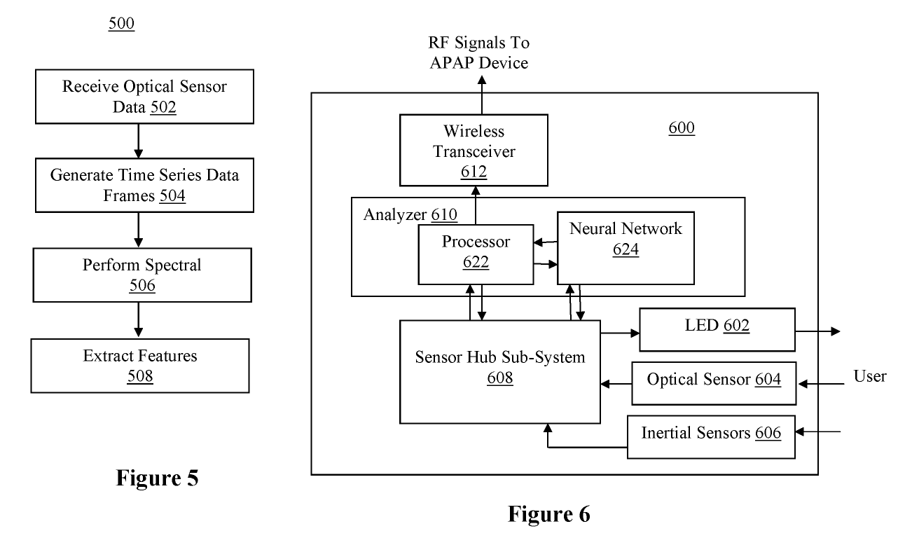

FIG. 5 illustrates an example process for processing optical sensor data, according to various embodiments. Process 500 for processing optical sensor data may include operations performed at blocks 502-508. The operations may be performed e.g., by a processor or controller of wearable device 100 or 200.

Process 500 may start at block 502. At block 502, optical sensor data outputted by optical sensors sensing lights reflected off the user/patient's capillaries may be received. Next, at block 504, the optical sensor data may be processed and time series data frames may be derived/generated from the optical sensor data. The analog signals of the heart rate measurements can be collected by the optical sensors, while analog signals of the motion measurement can be collected by the inertial sensors. The signals are then converted into digital samples by the analog-to-digital converter circuity to form the time series frames.

At 506, heart rate variability is obtained by computing the R-R intervals of the heart rate samples. Then the power spectrum of the frames of the HRV time series is computed, followed by the filtering the spectrum with different band-pass filters, and comparing the energy in the low frequency band (0.002 Hz to 0.5 Hz), the very low frequency (0.01 Hz to 0.05 Hz). Next at 508, features such as the ratios between spectral densities at different frequency bands are extracted to be used as inputs to the neural network for training and classification of the various sleep apnea conditions.

Referring now to FIG. 6, wherein a component view of an example wearable device suitable for practicing the present disclosure, according to various embodiments, is illustrated. As shown, in embodiments, wearable device 600 may include one or more LEDs 602, one or more optical sensors 604, one or more inertial sensors 606, sensor hub sub-system 608, analyzer 610 and wireless transceiver 612, coupled with each other. In embodiments, wearable device 600 may be wrist worn device 100 or 200.

As described earlier, one or more LEDs 602 may be configured to project lights on the capillaries of the user/patient. Optical sensors 604 may be configured to sense the light reflected off the capillaries of the user/patient. Inertial sensors 606 may be configured to various motion data that may depict sleep pattern of the user/patient, e.g., accelerometer and gyro sensors configured to sense motion of the user/patient. Sensor hub sub-system 608 may be configured to sample, condition, digitize and/or aggregate the optical and/or inertial sensor signals into optical and/or inertial sensor data. Except for their novel use to contribute in enhancement the sleep apnea therapy provided by a APAP device, LEDs 602, optical sensors 604, inertial sensors 606, and sensor hub sub-system may be any one of such elements known in the art.

Analyzer 610 may be configured to process and analyze the heart rate and motion samples to determine an apnea condition of the user/patient. In embodiments, analyzer 610 may include processor 622 and neural network 624. Processor 622 may be configured (e.g., programmed with executable instructions) to perform the operation of process 500 to process the heart rate and motion samples to generate the HRV data, and calculate the power spectral estimates. Processor 622 may further extract the features from the HRV such as the ration between spectral densities of various frequency sub-bands. Neural network 624 may be pre-trained to process the extracted features to recognize whether the features depict a mild, moderate, severe apnea or no apnea condition. Processor 622 and neural network 624, except for the logic to practice the present disclosure, may be anyone of such elements known in the art. In particular, processor 622 may be a controller, a single core or a multi-core processor.

Wireless transceiver 612 may be configured to transmit wireless signals encoded with the determined apnea conditions to the APAP device. In embodiments, wireless transceiver 612 may also be configured to transmit HRV data and/or other sleep patterns to the APAP device to allow the APAP device to perform fine grain setting adjustments in response to the determined apnea condition. In embodiments, wireless transceiver 612 may be a Bluetooth®, Near Field Communication (NFC), WiFi or other similar transceiver.

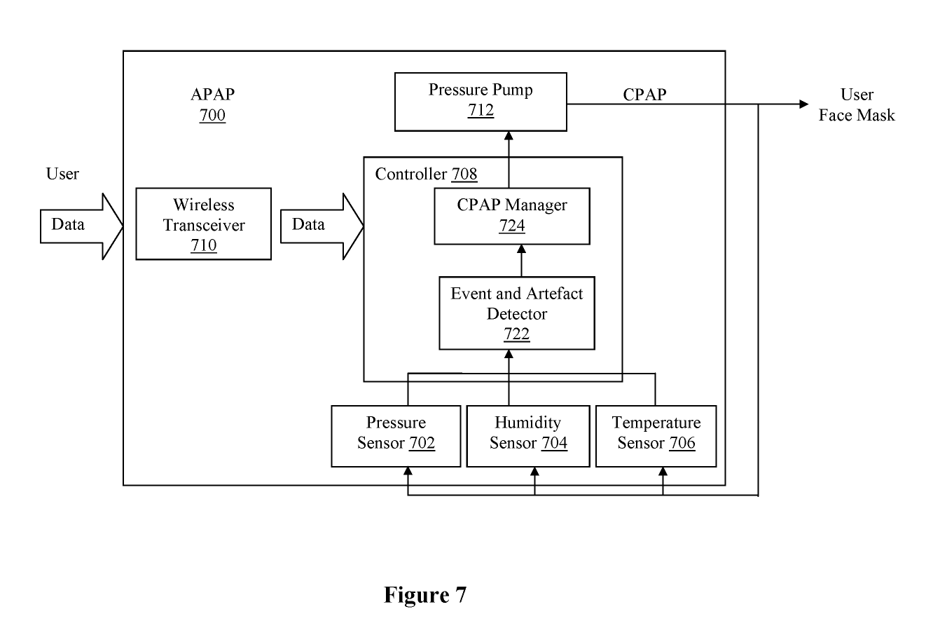

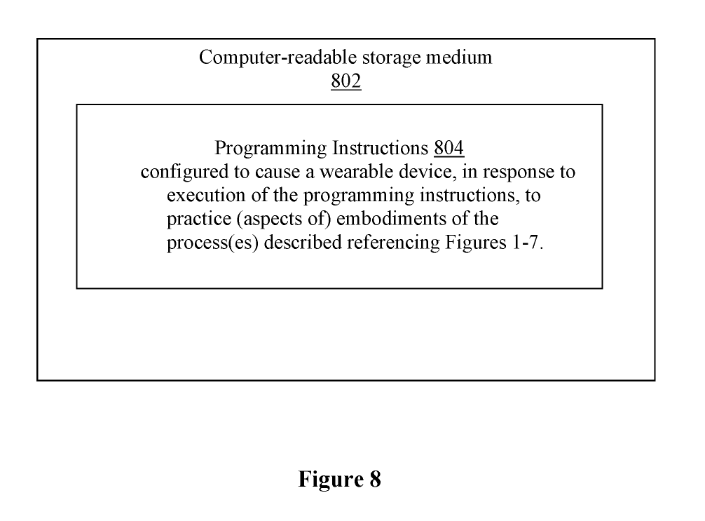

Referring now to FIG. 7, wherein a component view of an example APAP device for practicing the present disclosure, according to various embodiments, is shown. As illustrated, in embodiments, APAP device 700 may comprise a number of sensors 702-706, controller 708, wireless transceiver 710, and pressure pump 712.

Pressure pump 712 may be configured to generate an air stream to provide continuous positive air pressure (CPAP) for the user/patient, under the control of controller 712. Pressure pump 712 may be any one of such elements known in the art.

Sensors 702-706 may include pressure sensor 702, humidity sensor 704 and temperature sensor 706 to respectively sense pressure, humidity and temperature of the air stream generated by pressure pump 712 to provide continuous positive air pressure (CPAP) for the user/patient, and output the sensed pressure, humidity and temperature of the air stream to controller 708. Sensors 702-706 may be any one of a number of such sensors known in the art. In alternate embodiments, more or less sensors may be included.

Wireless transceiver 710 may be configured to receive wireless signals encoded with the determined apnea conditions from wearable device 100, 200 or 600. In embodiments, wireless transceiver 710 may also be configured to receive apnea conditions, HRV data and/or other sleep patterns from wearable device 100, 200 or 600. In embodiments, wireless transceiver 710 may be a Bluetooth®, Near Field Communication (NFC), WiFi or other similar transceiver.

Controller 708 may be configured to adaptively control pressure pump 712, based at least in part on the received apnea condition from wearable device 100, 200 or 600. In embodiments, controller 708 may be configured to adaptively control pressure pump 712, further based on the pressure, humidity and/or temperature sensed by sensors 702-706. In embodiments, controller 708 may be configured to perform fine grain modification to the settings of presume pump 712 to change the pressure, humidity and/or temperature of the air stream, based at least in part on apnea conditions, HRV data and/or other received from wearable device 100, 200, or 600.

In embodiments, controller 708 may include event and artefact detector 722 and CPAP manager 724. Event and artefact detector 722 may be configured to receive, process, and detect incongruence between the received apnea condition and the sensed pressure, humidity and/or temperature of the air stream, and output detection of events/exceptions for CPAP manager 724. CPAP manager 724 may be configured to receive HRV data and/or other sleep patterns from wearable device 100, 200 or 600, and events/exceptions detected by event and artefact detector 722, and in response, modify the settings of pressure pump 712 to adapt, customize, and/or enhance the air stream generated to provide CPAP for the user/patient.

In embodiments, for example, due to air leakage or other conditions, the APAP pressure setting without the data from the wearable device may suggest to set the air pressure to bet set at 10 cm H2O. The wearable device 600, after analyzing the HRV and sleep patterns, may suggest the pressure setting be 12 cm H2O. In this case, the CPAP manager 724 and event and artefact detector 722, may incorporate the information from the wearable device 600 and change the pressure setting to 11 cm H2O.

FIG. 8 illustrates an example computer-readable non-transitory storage medium that may be suitable for use to store instructions that cause an apparatus, in response to execution of the instructions by the apparatus, to practice selected aspects of the present disclosure. As shown, non-transitory computer-readable storage medium 802 may include a number of programming instructions 804. Programming instructions 804 may include instructions that are configured to enable a wearable device, e.g., wearable device 100, 200, or 600, in response to execution of the programming instructions, to provide an analyzer 610 that practices process 500. In embodiments, programming instructions 804 may further include instructions that are configured to enable a APAP device, e.g., APAP device 700, in response to execution of the programming instructions, to provide a controller 708 that practices the operations earlier described. In alternate embodiments, programming instructions 804 may be disposed on multiple computer-readable non-transitory storage media 802 instead. In still other embodiments, programming instructions 804 may be disposed on computer-readable transitory storage media 802, such as, signals.

Any combination of one or more computer usable or computer readable medium(s) may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to wireless, wireline, optical fiber cable, RF, etc.

Computer program code for carrying out operations of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's wearable device, partly on the user's wearable device, as a stand-alone software package, partly on the user's wearable device and partly on a remote computer or entirely on the remote computer or server. In the latter scenario, the remote computer may be connected to the user's wearable device through any type of network, including a local area network (LAN) or a wide area network (WAN), or the connection may be made to an external computer (for example, through the Internet using an Internet Service Provider).

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a,” “an” and “the” are intended to include plural forms as well, unless the context clearly indicates otherwise. It will be further understood that the terms “comprises” and/or “comprising,” when used in this specification, specific the presence of stated features, integers, steps, operations, elements, and/or components, but do not preclude the presence or addition of one or more other features, integers, steps, operation, elements, components, and/or groups thereof

The corresponding structures, material, acts, and equivalents of all means or steps plus function elements in the claims below are intended to include any structure, material or act for performing the function in combination with other claimed elements are specifically claimed. The description of the present disclosure has been presented for purposes of illustration and description, but is not intended to be exhaustive or limited to the disclosure in the form disclosed. Many modifications and variations will be apparent to those of ordinary skill without departing from the scope and spirit of the disclosure. The embodiment was chosen and described in order to best explain the principles of the disclosure and the practical application, and to enable others of ordinary skill in the art to understand the disclosure for embodiments with various modifications as are suited to the particular use contemplated.

Referring back to FIG. 6, for one embodiment, at least one of processors 622 may be packaged together with memory having programming instructions that provide an analyzer 610. For one embodiment, at least one of processors 622 may be packaged together with memory having programming instructions that provide an analyzer 610, to form a System in Package (SiP). For one embodiment, at least one of processors 622 may be integrated on the same die with memory having programming instructions that provide an analyzer 610. For one embodiment, at least one of processors 622 may be packaged together with memory having programming instructions that provide an analyzer 610, to form a System on Chip (SoC).

Further, in some embodiments, aspects or the entirety of analyzer 610 may be implemented in APAP 700 instead. That is, for these embodiments, wearable device 600 may be configured to collect and send sensed light and/or motion data to APAP 700; and APAP 700 may assume the functions of processing the sensed light and/or motion data, extract the heart variability and/or sleep patterns data, and determine the apnea condition. Other redistribution of functions are also possible.

Thus various example embodiments of the present disclosure have been described including, but are not limited to:

Example 1 may be a wearable apparatus, comprising: one or more light sources to project light onto a user; one or more optical sensors to sense light reflected off the user; an analyzer coupled with the one or more optical sensors to receive, process and analyze sensed light data for at least features of heart rate variability signals to determine an apnea condition of the user; wherein at least the determined apnea condition of the user is used to control an automatic positive airway pressure (APAP) device for the user.

Example 2 may be example 1, wherein the one or more light sources may comprise one or more light emitting diodes (LED).

Example 3 may be example 1, further comprising one or more inertial sensors to sense motion data of the user, wherein the analyzer is further coupled to the one or more inertial sensors to further receive, process and analyze the sensed motion data for sleep patterns of the user.

Example 4 may be example 1, wherein the analyzer may comprise a processor to receive and process the sensed light to extract the features of the heart rate variability or power spectral density data.

Example 5 may be example 4, wherein the processor may receive the sensed light data, process the received sensed light data into time series data frames, and perform spectral analysis on the time series data frames to extract the features of the heart rate variability, or power spectral density data.

Example 6 may be example 4, wherein the analyzer may further comprise a neural network coupled to the processor to receive and analyze the extracted features of the heart rate variability or power spectral density data to determine the apnea condition of the user.

Example 7 may be example 1, further comprising a wireless transceiver coupled with the analyzer to send the determined apnea condition, the heart rate variability data or sleep patterns data to the APAP device.

Example 8 may be any one of examples 1-7, wherein the apparatus is a wrist wearable device.

Example 9 may be an automatic positive airway pressure (APAP) device, comprising: a wireless transceiver arrangement to receive sensed light data of a user, or an apnea condition, heart rate variability or power spectral density data of the user determined or derived based on the sensed light data of the user; a pressure pump to output an air stream for the user, to provide continuous positive airwave pressure to the user; and a controller coupled to the wireless transceiver arrangement and the pressure pump to control the continuous positive airwave pressure provided by the pressure pump, based at least in part on the apnea condition, heart rate variability or power spectral density data of the user.

Example 10 may be example 9, wherein the wireless transceiver arrangement may receive the apnea condition, heart rate variability or power spectral density data of the user from a wearable device worn by the user.

Example 11 may be example 10, wherein the wearable device worn by the user may include: one or more light sources to project light onto the user; one or more optical sensors to sense light reflected off the user to provide the sensed light data; an analyzer coupled with the one or more optical sensors to receive, process and analyze the sensed light data for at least features of heart rate variability or power spectral density data to determine the apnea condition of the user; and a wireless transmitter coupled to the analyzer to transmit the determined apnea condition, the heart rate variability or the power spectral density data of the user to the APAP device.

Example 12 may be any one of example 10 or 11, further comprising one or more sensors to sense pressure, humidity or temperature of the air stream, and output sensed data indicative of the pressure, humidity or temperature condition of the air stream.

Example 13 may be example 12, wherein the controller may comprise a detector to detect the pressure, humidity or temperature condition of the air stream.

Example 14 may be example 13, wherein the controller may further comprise a manager coupled to the wireless transceiver arrangement and the detector to receive the apnea condition, heart rate variability, or power spectral density, and the detected pressure, humidity or temperature condition, from the wireless transceiver arrangement or the detector, and manage the pressure pump, based at least in part on the received apnea condition, heart rate variability, or power spectral density, and the detected pressure, humidity or temperature condition.

Example 15 may be example 9, wherein the wireless transceiver arrangement may further receive sleeping patterns of the user; and the controller may further control the continuous positive airwave pressure provided by the pressure pump, based on the received sleeping patterns of the user.

Example 16 may be example 9, wherein the wireless transceiver arrangement may receive the sensed light data of a user from a wearable device worn by the user; and the APAP device may further comprise an analyzer to receive, process and analyze the sensed light data for at least features of heart rate variability, or power spectral density signals to determine the apnea condition of the user.

Example 17 may be example 16, wherein the analyzer may comprise a processor to receive and process the sensed light data to extract the features of the heart rate variability or power spectral density data; and a neural network coupled to the processor to receive and analyze the extracted features of the heart rate variability or power spectral density signals to determine the apnea condition of the user.

Example 18 may be example 17, wherein the processor may receive the sensed light data, process the received the sensed light data into time series data frames, perform spectral analysis on the time series data frames to extract the features of the heart rate variability or power spectral density signals.

Example 19 may be a method for providing continuous positive airway pressure, comprising: sensing light reflected off a user; outputting sensed light data in response to the sensing; processing the sensed light data for at least features of heart rate variability or power spectral density; and analyzing the at least features of heart rate variability or power spectral density to determine an apnea condition of the user; wherein at least the determined apnea condition of the user is used to control an automatic positive airway pressure (APAP) device for the user.

Example 20 may be example 19, further comprising receiving motion data sensed by one or more inertial sensors; processing and analyzing the sensed motion data for sleep patterns of the user.

Example 21 may be example 19, wherein processing may comprise processing the sensed light to extract the features of the heart rate variability or power spectral density data.

Example 22 may be example 21, wherein processing the sensed light data may comprise processing the received sensed light data into time series data frames, and performing spectral analysis on the time series data frames to extract the features of the heart rate variability, or power spectral density data.

Example 23 may be example 21, wherein processing may further comprise analyzing the extracted features of the heart rate variability or power spectral density data to determine the apnea condition of the user.

Example 24 may be any one of examples 19-23, further comprising sending the determined apnea condition, the heart rate variability data or sleep patterns data to the APAP device.

Example 25 may be one or more computer-readable media comprising instructions that cause a device, in response to execution of the instructions by the device, to: process sensed light data associated with light reflected off a user for at least features of heart rate variability or power spectral density; and analyze the at least features of heart rate variability or power spectral density to determine an apnea condition of the user; wherein at least the determined apnea condition of the user is used to control an automatic positive airway pressure (APAP) device for the user.

Example 26 may be example 25, wherein the device is further caused to receive motion data sensed by one or more inertial sensors; process and analyze the sensed motion data for sleep patterns of the user.

Example 27 may be example 25, wherein to process may comprise to process the sensed light to extract the features of the heart rate variability or power spectral density data.

Example 28 may be example 27, wherein to process the sensed light data may comprise to process the received sensed light data into time series data frames, and perform spectral analysis on the time series data frames to extract the features of the heart rate variability, or power spectral density data.

Example 29 may be example 27, wherein to process may further comprise to analyze the extracted features of the heart rate variability or power spectral density data to determine the apnea condition of the user.

Example 30 may be any one of examples 25-29, wherein the device is further caused to send the determined apnea condition, the heart rate variability data or sleep patterns data to the APAP device.

Example 31 may be an apparatus for providing or assisting in provision of continuous positive airwave pressure to a user, comprising: means for processing sensed light data associated with light reflected off the user for at least features of heart rate variability or power spectral density; and means for analyzing the at least features of heart rate variability or power spectral density to determine an apnea condition of the user; wherein at least the determined apnea condition of the user is used to control an automatic positive airway pressure (APAP) device for the user.

Example 32 may be example 31, further comprising means for receiving motion data sensed by one or more inertial sensors; and means for processing and analyzing the sensed motion data for sleep patterns of the user.

Example 33 may be example 31, wherein means for processing may comprise means for processing the sensed light to extract the features of the heart rate variability or power spectral density data.

Example 34 may be example 33, wherein means for processing the sensed light data may comprise means for processing the received sensed light data into time series data frames, and means for performing spectral analysis on the time series data frames to extract the features of the heart rate variability, or power spectral density data.

Example 35 may be example 33, wherein means for processing may further comprise means for analyzing the extracted features of the heart rate variability or power spectral density data to determine the apnea condition of the user.

Example 36 may be example 31-35, further comprising means for sending the determined apnea condition, the heart rate variability data or sleep patterns data to the APAP device.

It will be apparent to those skilled in the art that various modifications and variations can be made in the disclosed embodiments of the disclosed device and associated methods without departing from the spirit or scope of the disclosure. Thus, it is intended that the present disclosure covers the modifications and variations of the embodiments disclosed above provided that the modifications and variations come within the scope of any claims and their equivalents.

What is claimed is:

1. A wearable apparatus, comprising:
one or more light sources to project light onto a user;
one or more optical sensors to sense light reflected off the user;
an analyzer coupled with the one or more optical sensors to receive, process and analyze sensed light data for at least features of heart rate variability signals to determine an apnea condition of the user;
wherein at least the determined apnea condition of the user is used to control an automatic positive airway pressure (APAP) device for the user.

2. The apparatus of claim 1, wherein the one or more light sources comprise one or more light emitting diodes (LED).

3. The apparatus of claim 1, further comprising one or more inertial sensors to sense motion data of the user, wherein the analyzer is further coupled to the one or more inertial sensors to further receive, process and analyze the sensed motion data for sleep patterns of the user.

4. The apparatus of claim 1, wherein the analyzer comprises a processor to receive and process the sensed light to extract the features of the heart rate variability or power spectral density data.

5. The apparatus of claim 4, wherein the processor is to receive the sensed light data, process the received sensed light data into time series data frames, and perform spectral analysis on the time series data frames to extract the features of the heart rate variability, or power spectral density data.

6. The apparatus of claim 4, wherein the analyzer further comprises a neural network coupled to the processor to receive and analyze the extracted features of the heart rate variability or power spectral density data to determine the apnea condition of the user.

7. The apparatus of claim 1, further comprising a wireless transceiver coupled with the analyzer to send the determined apnea condition, the heart rate variability data or sleep patterns data to the APAP device.

8. The apparatus of claim 1, wherein the apparatus is a wrist wearable device.

9. An automatic positive airway pressure (APAP) device, comprising:
a wireless transceiver arrangement to receive sensed light data of a user, or an apnea condition, heart rate variability or power spectral density data of the user determined or derived based on the sensed light data of the user;
a pressure pump to output an air stream for the user, to provide continuous positive airwave pressure to the user; and
a controller coupled to the wireless transceiver arrangement and the pressure pump to control the continuous positive airwave pressure provided by the pressure pump, based at least in part on the apnea condition, heart rate variability or power spectral density data of the user.

10. The APAP device of claim 9, wherein the wireless transceiver arrangement is to receive the apnea condition, heart rate variability or power spectral density data of the user from a wearable device worn by the user.

11. The APAP device of claim 10, wherein the wearable device worn by the user includes:
one or more light sources to project light onto the user;
one or more optical sensors to sense light reflected off the user to provide the sensed light data;
an analyzer coupled with the one or more optical sensors to receive, process and analyze the sensed light data for at least features of heart rate variability or power spectral density data to determine the apnea condition of the user; and
a wireless transmitter coupled to the analyzer to transmit the determined apnea condition, the heart rate variability or the power spectral density data of the user to the APAP device.

12. The APAP device of claim 10, further comprising one or more sensors to sense pressure, humidity or temperature of the air stream, and output sensed data indicative of the pressure, humidity or temperature condition of the air stream.

13. The APAP device of claim 12, wherein the controller comprises a detector to detect the pressure, humidity or temperature condition of the air stream.

14. The APAP device of claim 13, wherein the controller further comprises a manager coupled to the wireless transceiver arrangement and the detector to receive the apnea condition, heart rate variability, or power spectral density, and the detected pressure, humidity or temperature condition, from the wireless transceiver arrangement or the detector, and manage the pressure pump, based at least in part on the received apnea condition, heart rate variability, or power spectral density, and the detected pressure, humidity or temperature condition.

15. The APAP device of claim 9, wherein the wireless transceiver arrangement is to further receive sleeping patterns of the user; and the controller is to further control the continuous positive airwave pressure provided by the pressure pump, based on the received sleeping patterns of the user.

16. The APAP device of claim 9, wherein the wireless transceiver arrangement is to receive the sensed light data of a user from a wearable device worn by the user; and the APAP device further comprises an analyzer to receive, process and analyze the sensed light data for at least features of heart rate variability, or power spectral density signals to determine the apnea condition of the user.

17. The APAP device of claim 16, wherein the analyzer comprises a processor to receive and process the sensed light data to extract the features of the heart rate variability or power spectral density data; and a neural network coupled to the processor to receive and analyze the extracted features of the heart rate variability or power spectral density signals to determine the apnea condition of the user.

18. The APAP device of claim 17, wherein the processor is to receive the sensed light data, process the received the sensed light data into time series data frames, perform spectral analysis on the time series data frames to extract the features of the heart rate variability or power spectral density signals.

19. A method for providing continuous positive airway pressure, comprising:
sensing light reflected off a user;
outputting sensed light data in response to the sensing;
processing the sensed light data for at least features of heart rate variability or power spectral density; and
analyzing the at least features of heart rate variability or power spectral density to determine an apnea condition of the user;
wherein at least the determined apnea condition of the user is used to control an automatic positive airway pressure (APAP) device for the user.

20. The method of claim 19, further comprising receiving motion data sensed by one or more inertial sensors; processing and analyzing the sensed motion data for sleep patterns of the user.

21. The method of claim 19, wherein processing comprises processing the sensed light to extract the features of the heart rate variability or power spectral density data; and wherein processing the sensed light data comprises processing the received sensed light data into time series data frames, and performing spectral analysis on the time series data frames to extract the features of the heart rate variability, or power spectral density data, and analyzing the extracted features of the heart rate variability or power spectral density data to determine the apnea condition of the user.

22. The method of claim 19, further comprising sending the determined apnea condition, the heart rate variability data or sleep patterns data to the APAP device.

23. One or more computer-readable media comprising instructions that cause a device, in response to execution of the instructions by the device, to:
process sensed light data associated with light reflected off a user for at least features of heart rate variability or power spectral density; and
analyze the at least features of heart rate variability or power spectral density to determine an apnea condition of the user;
wherein at least the determined apnea condition of the user is used to control an automatic positive airway pressure (APAP) device for the user.

24. The computer-readable media of claim 24, wherein the device is further caused to receive motion data sensed by one or more inertial sensors; process and analyze the sensed motion data for sleep patterns of the user, and to process comprises to process the sensed light to extract the features of the heart rate variability or power spectral density data.

25. The computer-readable media of claim 24, wherein to process the sensed light data comprises to process the received sensed light data into time series data frames, and perform spectral analysis on the time series data frames to extract the features of the heart rate variability, or power spectral density data; and wherein to process further comprises to analyze the extracted features of the heart rate variability or power spectral density data to determine the apnea condition of the user.
